SEQUENCE LISTING (6 sequences)

SEQ ID NO: 1 - protein, 552 aa, Artificial Sequence
  Synthetic
MRLLPLAPGRLRRGSPRHLPSCSPALLLLVLGGCLGVFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLL

SEQ ID NO: 2 - protein, 572 aa, Artificial Sequence
  Synthetic
MRLLPLAPGRLRRGSPRHLPSCSPALLLLVLGGCLGVFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLLGGGGSGGGGSEQKLISEEDL

SEQ ID NO: 3 - DNA, 1656 bp, Artificial Sequence
  Synthetic
atgcgactgctgcccctggctcccggtagactgcgaagaggttcaccacgacatctgccaagttgctcccccgccctgctgctgctggtgctgggcggatgcctgggagtctttggagtggcagctggcacacggaggccaaacgtggtcctgctgctgactgacgatcaggacgaggtgctggggggtatgacccccctgaagaaaacaaaggccctgatcggtgaaatgggcatgacattcagctccgcatacgtgcctagcgccctgtgctgtccaagccgggcttccattctgactgggaaatatccacacaatcaccatgtggtcaacaataccctggagggcaactgctctagtaagtcttggcagaaaatccaggaacctaatacatttccagccattctgaggagtatgtgcggttaccagactttctttgctggcaagtacctgaacgagtatggagctccagatgcaggaggactggaacacgtgccactgggatggtcctactggtatgccctggagaagaacagcaaatactataactacaccctgtctatcaatggaaaggctagaaaacatggggaaaactactctgtggactatctgacagatgtcctggcaaatgtgagtctggactttctggattacaagtcaaacttcgagcccttctttatgatgattgccactccagctcctcactctccctggaccgcagcaccacagtatcagaaggctttccagaacgtgttcgcacctaggaacaaaaatttcaacatccacggaaccaacaagcattggctgattagacaggcaaaaaccccaatgacaaattcaagcatccagttcctggacaacgcctttcgaaagcggtggcagaccctgctgagcgtggacgatctggtggagaagctggtcaaacgcctggagtttacaggggaactgaacaacacttacattttctataccagcgataacggataccatactgggcagttttccctgcccatcgacaagcgacagctgtatgagttcgatatcaaggtgcccctgctggtccggggaccagggatcaagcctaatcagactagtaaaatgctggtggctaacattgacctggggcccaccatcctggacattgcaggttacgatctgaacaagacacagatggatggcatgtccctgctgcctatcctgcggggagcctcaaatctgacctggaggagcgacgtgctggtcgagtatcagggtgaaggccgcaacgtgactgatccaacctgcccctccctgtctccaggcgtgtcacagtgttttcctgactgcgtctgtgaggatgcctacaacaatacctatgcttgcgtgcgaacaatgagcgccctgtggaacctgcagtactgtgagttcgacgatcaggaggtgtttgtcgaagtgtataatctgacagcagaccctgatcagatcacaaacattgccaagactatcgacccagagctgctggggaaaatgaattacagactgatgatgctgcagagttgctcaggtcctacatgtcgcactccaggcgtgttcgaccccggctataggtttgatcctagactgatgttctctaaccgcggaagtgtccgaaccagacgcttctccaagcatctgctg

SEQ ID NO: 4 - DNA, 1656 bp, Artificial Sequence
  Synthetic
atgcggctcctgcctctagccccaggtcggctccggcggggcagcccccgccacctgccctcctgcagcccagcgctgctactgctggtgctgggcggctgcctgggggtcttcggggtggctgcgggaacccggaggcccaacgtggtgctgctcctcacggacgaccaggacgaagtgctcggcggcatgacaccgctaaagaaaaccaaagctctcatcggagagatggggatgactttttccagtgcttatgtgccaagtgctctctgctgccccagcagagccagtatcctgacaggaaagtacccacataatcatcacgttgtgaacaacactctggaggggaactgcagtagtaagtcctggcagaagatccaagaaccaaatactttcccagcaattctcagatcaatgtgtggttatcagaccttttttgcagggaaatatttaaatgagtacggagccccagatgcaggtggactagaacacgttcctctgggttggagttactggtatgccttggaaaagaattctaagtattataattacaccctgtctatcaatgggaaggcacggaagcatggtgaaaactatagtgtggactacctgacagatgttttggctaatgtctccttggactttctggactacaagtccaactttgagcccttcttcatgatgatcgccactccagcgcctcattcgccttggacagctgcacctcagtaccagaaggctttccagaatgtctttgcaccaagaaacaagaacttcaacatccatggaacgaacaagcactggttaattaggcaagccaagactccaatgactaattcttcaatacagtttttagataatgcatttaggaaaaggtggcaaactctcctctcagttgatgaccttgtggagaaactggtcaagaggctggagttcactggggagctcaacaacacttacatcttctatacctcagacaatggctatcacacaggacagttttccttgccaatagacaagagacagctgtatgagtttgatatcaaagttccactgttggttcgaggacctgggatcaaaccaaatcagacaagcaagatgctggttgccaacattgacttgggtcctactattttggacattgctggctacgacctaaataagacacagatggatgggatgtccttattgcccattttgagaggtgccagtaacttgacctggcgatcagatgtcctggtggaataccaaggagaaggccgtaacgtcactgacccaacatgcccttccctgagtcctggcgtatctcaatgcttcccagactgtgtatgtgaagatgcttataacaatacctatgcctgtgtgaggacaatgtcagcattgtggaatttgcagtattgcgagtttgatgaccaggaggtgtttgtagaagtctataatctgactgcagacccagaccagatcactaacattgctaaaaccatagacccagagcttttaggaaagatgaactatcggttaatgatgttacagtcctgttctgggccaacctgtcgcactccaggggtttttgaccccggatacaggtttgacccccgtctcatgttcagcaatcgcggcagtgtcaggactcgaagattttccaaacatcttctg

SEQ ID NO: 5 - protein, 579 aa, Artificial Sequence
  Synthetic
MRLLPLAPGRLRRGSPRHLPSCSPALLLLVLGGCLGVFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLLGGENLYFQGGGSGGGGSEQKLISEEDL

SEQ ID NO: 6 - protein, 560 aa, Artificial Sequence
  Synthetic
MRLLPLAPGRLRRGSPRHLPSCSPALLLLVLGGCLGVFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLLGGENLYFQ

(19) United States
(12) Patent Application Publication
(10) Pub. No.: US 2021/0015906 A1
(43) Pub. Date: Jan. 21, 2021

(54) ENZYME REPLACEMENT THERAPY FOR MUCOPOLYSACCHARIDOSIS IIID

(71) Applicant: Los Angeles Biomedical Research Institute at Harbor-UCLA Medical Center, Torrance, CA (US)

(72) Inventors: Patricia Dickson, Torrance, CA (US); Tsui-Fen Chou, Torrance, CA (US); Sean Ekins, Fuquay-Varina, NC (US); Shih-Hsin Kan, Torrance, CA (US); Steven Le, Torrance, CA (US); Derek R. Moen, Hermosa Beach, CA (US)

(21) Appl. No.: 16/919,104

(22) Filed: Jul. 1, 2020

Related U.S. Application Data
(63) Continuation of application No. 15/946,505, filed on Apr. 5, 2018, now abandoned.
Application No. 15/946,505 is a continuation-in-part of application No. PCT/US2016/055822, filed on Oct. 6, 2017.
(60) Provisional application No. 62/238,024, filed on Oct. 6, 2015.
(60) Provisional application No. 62/611,472, filed on Dec. 28, 2017.

(51) Int. Cl. A61K 38/47; A61P 43/00; A61K 47/66; A61K 9/00

(52) U.S. Cl. CPC A61K 38/47; A61P 43/00; A61K 31/352; A61K 9/0019; A61K 47/67

(57) ABSTRACT

The present disclosure relates to compositions and methods for treating Sanfilippo syndrome (also known as Sanfilippo disease type D, Sanfilippo D, mucopolysaccharidosis type IIID, MPS IIID). The method can entail injecting to the spinal fluid of a MPS IIID patient an effective amount of a composition comprising a recombinant human acetylglucosamine-6-sulfatase (GNS) protein comprising the amino acid sequence of SEQ ID NO: 1 or an amino acid sequence having at least 90% sequence identity to SEQ ID NO: 1 and having the enzymatic activity of the human GNS protein. The composition can be provided in an artificial cerebrospinal fluid. About 1 mg to about 100 mg of the recombinant polypeptide may be administered to the patient once every 2 weeks to 6 months.

### CROSS REFERENCE TO RELATED APPLICATIONS

This application is a continuation of U.S. patent application Ser. No. 15/946,505, filed Apr. 5, 2018, which is a continuation-in-part of International Application No. PCT/US2016/055822, filed Oct. 6, 2016, which claims the benefit under 35 U.S.C. § 119(e) of U.S. Provisional Application Ser. No. 62/238,024, filed Oct. 6, 2015. This application also claims the benefit under 35 U.S.C. § 119(e) of U.S. Provisional Application Ser. No. 62/611,472, filed Dec. 28, 2017. The contents of these earlier-filed application are incorporated by reference in their entirety into the present disclosure.

### STATEMENT AS TO FEDERALLY SPONSORED RESEARCH OR DEVELOPMENT

This invention was made with government support under R41 NS089061-01 awarded by National Institute of Neurological Disorders and Stroke at National Institute of Health. The government has certain rights in the invention.

### SEQUENCE LISTING

The instant application contains a Sequence Listing which has been submitted in ASCII format via EFS-Web and is hereby incorporated by reference in its entirety. The ASCII copy, created on Apr. 5, 2018, is named 223143_ST25.txt and is 24,667 bytes in size.

### BACKGROUND

Sanfilippo disease (mucopolysaccharidosis type III; MPS III) is a devastating neurodegenerative lysosomal storage disorder of childhood. Babies appear normal at birth, learn to walk and talk, but then gradually, progressively, deteriorate to a vegetative state over the span of 10 or 20 years. The central pathologic features of MPS III are neurologic: there is a slowing of development, severe behavioral problems, progressive cognitive decline, dementia, and decline in motor skills that steadily lead to immobility, unresponsiveness, and death.

The fundamental cause of MPS III is an inherited mutation in one of the 4 enzymes required to catabolize heparan sulfate (HS), a glycosaminoglycan which plays important structural and functional roles in the brain and elsewhere. Each type of MPS III (A to D) is due to deficiency of a different enzyme in the HS breakdown pathway. Because MPS III is rare and affects the brain (which is difficult to treat), motivation for pharmaceutical and biotechnology companies to develop new therapies has been limited.

There is no cure or effective treatment available for MPS IIID, and there is therefore an unmet need for developing such a treatment.

### SUMMARY

The present disclosure provides methods and compositions of treating Sanfilippo syndrome (also known as Sanfilippo disease type D, Sanfilippo D, mucopolysaccharidosis type IIID, MPS IIID) by, e.g., intrathecal (IT) administration of an alpha-N-acetylglucosamine-6-sulfatase (GNS) protein. A suitable GNS protein can be a recombinant, gene-activated or natural protein. In some embodiments, a suitable GNS protein is a recombinant GNS protein. In some embodiments, a recombinant GNS protein is a protein containing a GNS domain and a lysosomal targeting moiety.

In one embodiment, the disclosure provides a method of treating mucopolysaccharidosis type IIID (MPS IIID) in a human patient in need thereof, comprising injecting to the spinal fluid of the patient an effective amount of a composition comprising a recombinant polypeptide comprising the amino acid sequence of SEQ ID NO: 1 or an amino acid sequence (a) having at least 95% sequence identity to SEQ ID NO: 1 and (b) having the enzymatic activity of GNS, wherein the composition is provided in an artificial cerebrospinal fluid.

In some embodiments, about 1 mg to about 100 mg of the recombinant polypeptide is administered to the patient each time (or each day when administration is conducted). In some embodiments, the administration is conducted once every 2 to 26 weeks (or every 2 weeks to every 6 months). In some embodiments, the recombinant polypeptide comprises the amino acid sequence of SEQ ID NO: 2, 5 or 6.

The artificial cerebrospinal fluid may have a pH of about 6 to 7.5, without limitation. In some embodiments, the artificial cerebrospinal fluid comprises about 130-170 mEq/1 sodium, about 2.5-5 mEq/1 potassium, about 1-3 mEq/1 calcium, about 0.5-3 mEq/1 magnesium, about 120-180 mEq/1 chloride, and about 0.5-2 mEq/1 phosphate. In some aspects, the artificial cerebrospinal fluid comprises about 140-160 mEq/1 sodium, about 3.5-4.5 mEq/1 potassium, about 2.5-3 mEq/1 calcium, about 2-3 mEq/1 magnesium, about 120-140 mEq/1 chloride, and about 1-2 mEq/1 phosphate. In some aspects, the artificial cerebrospinal fluid comprises about 140-160 mEq/1 sodium, about 3.5-4.5 mEq/1 potassium, about 2.5-3 mEq/1 calcium, about 2-3 mEq/1 magnesium, about 120-140 mEq/1 chloride, about 1-2 mEq/1 phosphate, about 18-25 mEq/1 bicarbonate, and about 2-3 mEq/1 sulfate. In some aspects, the artificial cerebrospinal fluid has an osmolarity of about 250-350 mOsm/1.

In some aspects, the recombinant polypeptide is has maximum enzymatic activity at a pH within 5.4 to 5.8. In some embodiments, the recombinant polypeptide enters a human fibroblast cell when the recombinant polypeptide is incubated with the human fibroblast cell. In some aspects, the composition comprises from about 0.5 mg to about 30 mg of the recombinant protein per ml of the artificial cerebrospinal fluid.

In some aspects, the recombinant polypeptide further comprises a lysosomal targeting moiety. In some aspects, the recombinant polypeptide is glycosylated, which glycosylation adds from 25 kDa to 45 kDa molecular weight to the recombinant polypeptide.

Combination therapies are also provided. In addition to the injection, the patient can further receive a therapy such as bone marrow replacement, or administration of genistein or a chaperone.

Also provided, in one embodiment, is a polynucleotide comprising the nucleic acid sequence of SEQ ID NO: 3 or a nucleic acid sequence (a) having at least 85% sequence identify to SEQ ID NO: 3, (b) encoding the amino acid sequence of SEQ ID NO: 1, and (c) having no more than 95% sequence identity to SEQ ID NO: 4. Further provided, in one embodiment, is a cell comprising the polynucleotide of the disclosure.

### BRIEF DESCRIPTION OF THE DRAWINGS

FIG. 1 shows an example amino acid sequence (SEQ ID NO: 1) useful for treating MPS IIID.

FIG. 2A shows the sequence of SEQ ID NO: 2 which, as compared to SEQ ID NO: 1, further includes a glycine-serine linker (underlined) and a c-myc tag (bold and italic) for protein purification.

FIG. 2B shows the sequence of SEQ ID NO: 5 which, as compared to SEQ ID NO: 2, further includes a cleavage site (bold and underlined) recognizable by the Tobacco Etch Virus (TEV) protease which is useful for removing the c-myc tag.

FIG. 2C shows the sequence of SEQ ID NO: 6, after the c-myc tag is removed from SEQ ID NO: 5 by a (TEV) protease.

FIG. 3 shows an illustrative cDNA sequence (SEQ ID NO: 3) for encoding a recombinant GNS protein of the present disclosure.

FIG. 4 shows the wild-type human cDNA sequence (SEQ ID NO: 4) for GNS.

FIG. 5 presents a sequence alignment between SEQ ID NO: 3 and 4.

FIG. 6A-D show that the purified recombinant human alpha-N-acetylglucosamine-6-sulfatase (rhGNS) was heavily glycosylated and enzymatically active. A) Western blot of rhGNS purification using antibodies against GNS and against the purification tag, myc. Non-clinical-grade rhGNS purchased from R&D systems and alpha-N-acetylglucosaminidase (NAGLU) produced from CHO cells were used as positive controls. B) PNGase F and Endo H treatment of purified rhGNS results in a shift in molecular weight, demonstrating that the protein is glycosylated. C) Michaelis-Menten Curves of rhGNS. Enzymatic activity of rhGNS was assayed using a fluorogenic substrate (4-MU-GNS) with a 4 h second step (squares) vs. 24 h second step (circles). Kₘ was 3.97 mM and Vmax was 336,359 nmol/24 h with the shorter assay. D) pH profile of rhGNS activity. Optimal assay conditions occurred within acidic pH range (4-6). Means and S.D. of triplicate experiments.

FIG. 7A-D show rhGNS entered human MPS IIID cells, targeted to lysosomes, and reduced GAG storage. A) Confocal microscopy of rhGNS uptake into MPS IIID human fibroblasts. Blue: DAPI, Green: rhGNS (anti-myc), Red: Lysotracker. Top row: treated with rhGNS. Bottom row, no rhGNS applied. B) rhGNS intracellular uptake and inhibition assay. Cell lysates from MPS IIID human fibroblasts were assayed for GNS activity following 4 hour treatment with rhGNS with or without 5 mM mannose-6-phosphate (M6P). C) Heparan sulfate GAG reduction in MPS IIID human fibroblasts treated with 150 ng/ml rhGNS for 72 h at 37° C. or untreated. Shown is a representative experiment from triplicate experiments. Means and S.D. of triplicate assays. Two wild-type (WT) fibroblast lines are shown as controls. D) Radiolabeled GAG accumulation measured at different concentrations of purified rhGNS (0-250 pM) demonstrated an exponential decrease in storage, with storage reduced by half (EC₅₀) at 5.5 pM. Radiolabeled GAGs were extracted and measured via scintillation counting, and radioactive counts per minute were normalized to protein concentration. EC₅₀ was calculated using exponential decay with a bottom of 40% (equal to WT levels). Means and S.D. of triplicate assays.

FIG. 8 presents a chart showing that rhGNS is active at body temperature (activity vs temperature, normalized to activity at 24° C.). Means and S.D. of triplicate experiments. Each point was assayed in duplicate.

FIG. 9 demonstrates rhGNS stability in artificial cerebrospinal fluid. Activity is normalized to activity at day=0. Means and S.D. of triplicate experiments. Each point was assayed in duplicate.

FIG. 10A-B show the GNS enzyme activity (nmol/hr/mg) in MPS IIID mice or control mice receiving different treatments.

FIGS. 11A and 11B show the activities of two lysosomal enzymes, alpha-N-acetylglucosaminidase (NAGLU) and β-hexoaminidase (HEX) one day after the MPS IIID or control mice receiving different treatments.

### DETAILED DESCRIPTION

### I. Definitions

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

The term “protein” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. Single letter and three letter abbreviations of the naturally occurring amino acids are listed below. A peptide of three or more amino acids is commonly called an oligopeptide if the peptide chain is short. If the peptide chain is long, the peptide is commonly called a polypeptide or a protein.

A “pharmaceutical composition” is intended to include the combination of an active agent with a carrier, inert or active, making the composition suitable for diagnostic or therapeutic use in vitro, in vivo or ex vivo.

“An effective amount” refers to the amount of derivative sufficient to induce a desired biological and/or therapeutic result. That result can be alleviation of the signs, symptoms, or causes of a disease, or any other desired alteration of a biological system. The effective amount will vary depending upon the specific recombinant GNS protein used, the dosing regimen of the recombinant GNS protein, timing of administration of the recombinant GNS protein, the subject and disease condition being treated, the weight and age of the subject, the severity of the disease condition, the manner of administration and the like, all of which can be determined readily by one of ordinary skill in the art.

As used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

“Treating” also covers any treatment of a disorder in a mammal, and includes: (a) preventing a disorder from occurring in a subject that may be predisposed to a disorder, but may have not yet been diagnosed as having it, e.g., prevent MPS IIID symptoms in a patient with the genetic features of the MPS IIID disease.

As used herein, to “treat” further includes systemic amelioration of the symptoms associated with the pathology and/or a delay in onset of symptoms. Clinical and sub-clinical evidence of “treatment” will vary with the pathology, the individual and the treatment.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. A “subject” of diagnosis or treatment is a cell or a mammal, including a human.

The agents and compositions of the present disclosure can be used in the manufacture of medicaments and for the treatment of humans and other animals by administration in accordance with conventional procedures, such as an active ingredient in pharmaceutical compositions.

An agent of the present disclosure can be administered for therapy by any suitable route, specifically by intrathecal (injection into the spinal fluid), intravenous or intranasal administration.

The terms “polynucleotide” and “oligonucleotide” are used interchangeably and refer to a polymeric form of nucleotides of any length, either deoxyribonucleotides or ribonucleotides or analogs thereof. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art.

For each polynucleotide or polypeptide disclosed in the present disclosure, its biological equivalents are also contemplated. Biologically equivalents are those having the specified percent sequence identity (e.g., at least 75%, 80%, 85%, 90%, 95%, 97%, 98%, 99%) and having the same or similar biological activity as the reference polypeptide or encoding a polypeptide that has the same or similar biological activity as the polypeptide encoded by the reference polynucleotide. In some embodiments, a biologically equivalent has one, two, three, four or five addition, deletion or substitution of amino acid resides or nucleotides as compared to the reference polypeptide or polynucleotide.

### II. Methods of Treating MPS IIID

The present disclosure provides, in one embodiment, an enzyme replacement treatment (ERT) for MPS IIID that will ameliorate or reverse the catastrophic and fatal neurologic decline caused by this disease. Unlike MPS I, the symptoms of MPS III are largely localized to the brain. Hence, an effective MPS III treatment needs to gain access to the brain. Delivery of large proteins such as the enzymes genetically missing in MPS III will not cross the blood-brain barrier if delivered systemically.

Therefore, in the present technology, a rhGNS is delivered intrathecally (directly into the spinal fluid) to effectively treat the underlying causes of the neurologic symptoms that dominate MPS III pathology.

Experimental data presented herein showed robust expression of rhGNS in Chinese hamster ovary cells enabling effective production of the protein. In a larger scale experiment, rhGNS ˜100 μg per 1500 mL media was produced in CHO cells, and was purified to a specific activity of ˜100,000 activity units/mg. The data further showed that the expressed rhGNS protein demonstrated maximal enzymatic activity at pH 5.6, demonstrated good enzymatic activity at 37° C. and was stable for over one month at 4° C. in artificial cerebrospinal fluid storage buffer.

Further, experiments showed intracellular enzymatic activity of rhGNS in MPS III fibroblasts when rhGNS was added to the media, and 70±6% rhGNS colocalized with lysosomal markers using confocal microscopy and confirmed that radiolabelled HS diminished 33-65% in MPS III fibroblasts treated with rhGNS (to wild-type levels).

Moreover, in a mouse MPS IIID model, when about 5.3 μg of the rhGNS was administered, GNS enzyme activity recovered to higher than normal levels within 2 hours following the administration, and the activities of two lysosomal enzymes, alpha-N-acetylglucosaminidase (NAGLU) and β-hexoaminidase (HEX) which are overexpressed in MPS IIID patients, were significantly reduced within 1 day after the treatment. Such high and fast-acting in vivo efficacy was surprising and unexpected.

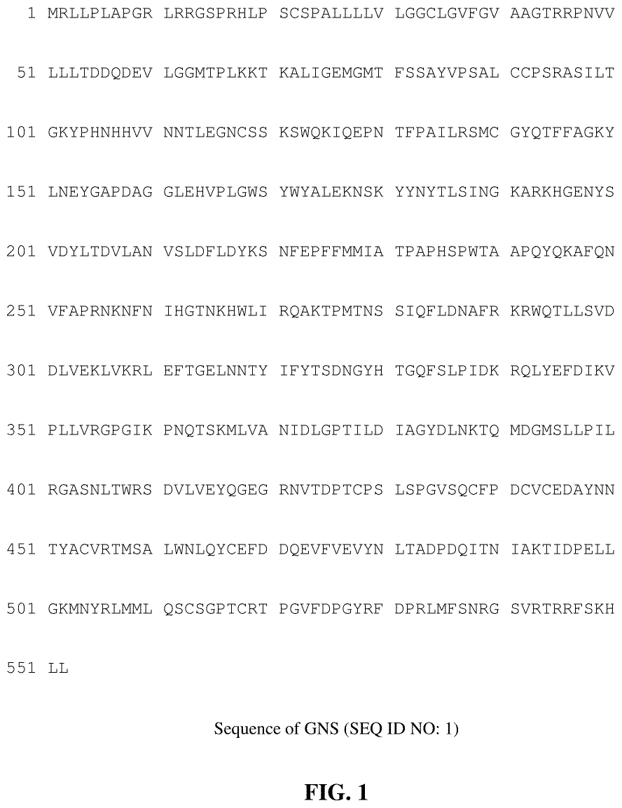

In accordance with one embodiment of the present disclosure, provided is a method of treating mucopolysaccharidosis type IIID (MPS IIID) in a human patient in need thereof. In one aspect, the method entails injecting to the spinal fluid of the patient an effective amount of a composition comprising a rhGNS protein. In one aspect, the rhGNS includes the amino acid sequence of SEQ ID NO: 1 or an amino acid sequence (a) having a sequence identity (e.g., at least 75%, 80%, 85%, 90%, 95%, 97%, 98%, or 99% sequence identity) to SEQ ID NO: 1 (see FIG. 1) and (b) having the enzymatic activity of human GNS protein. In one aspect, the rhGNS includes the amino acid sequence of SEQ ID NO: 2 or an amino acid sequence (a) having a sequence identity (e.g., at least 75%, 80%, 85%, 90%, 95%, 97%, 98%, or 99% sequence identity) to SEQ ID NO: 2 and (b) having the enzymatic activity of human GNS protein. In one aspect, the rhGNS includes the amino acid sequence of SEQ ID NO: 5 or 6, or an amino acid sequence (a) having a sequence identity (e.g., at least 75%, 80%, 85%, 90%, 95%, 97%, 98%, or 99% sequence identity) to SEQ ID NO: 5 or 6 and (b) having the enzymatic activity of human GNS protein.

In some embodiments, the rhGNS can be administered once every two weeks to every six months. In one embodiment, the rhGNS can be administered once every two weeks, every three weeks, every four weeks, every five weeks, every six weeks, every seven weeks, every eight weeks, every month, every 2 months, every 3 months, every 4 months, every 5 months, or every 6 months. In some embodiments, the rhGNS is administered for at least 1 year, 2 years, 5 years, or 10 years. In some embodiments, the rhGNS is administered for no more than 2 years, 3 years, 4 year, 5 years, 10 years, 15 years, 20 years or 25 years.

In some embodiments, for each administration, the amount of rhGNS is from about 0.05 mg/kg to about 5 mg/kg. In some embodiments, for each administration, the amount of rhGNS is from about 0.05 mg/kg to about 4 mg/kg, from about 0.05 mg/kg to about 3 mg/kg, from about 0.05 mg/kg to about 2 mg/kg, from about 0.05 mg/kg to about 1 mg/kg, from about 0.05 mg/kg to about 0.5 mg/kg, from about 0.05 mg/kg to about 0.25 mg/kg, from about 0.1 mg/kg to about 4 mg/kg, from about 0.15 mg/kg to about 4 mg/kg, from about 0.20 mg/kg to about 4 mg/kg, from about 0.25 mg/kg to about 4 mg/kg, from about 0.5 mg/kg to about 4 mg/kg, from about 0.1 mg/kg to about 1 mg/kg, from about 0.1 mg/kg to about 0.9 mg/kg, from about 0.1 mg/kg to about 0.8 mg/kg, from about 0.1 mg/kg to about 0.7 mg/kg, from about 0.1 mg/kg to about 0.6 mg/kg, from about 0.1 mg/kg to about 0.5 mg/kg, from about 0.1 mg/kg to about 0.4 mg/kg, from about 0.1 mg/kg to about 0.3 mg/kg, from about 0.2 mg/kg to about 1 mg/kg, from about 0.2 mg/kg to about 0.9 mg/kg, from about 0.2 mg/kg to about 0.8 mg/kg, from about 0.2 mg/kg to about 0.7 mg/kg, from about 0.2 mg/kg to about 0.6 mg/kg, from about 0.2 mg/kg to about 0.5 mg/kg, from about 0.2 mg/kg to about 0.4 mg/kg. In some embodiments, for each administration, the amount of rhGNS is about 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.11 mg/kg, 0.12 mg/kg, 0.13 mg/kg, 0.14 mg/kg, 0.15 mg/kg, 0.16 mg/kg, 0.17 mg/kg, 0.18 mg/kg, 0.19 mg/kg, 0.2 mg/kg, 0.21 mg/kg, 0.22 mg/kg, 0.23 mg/kg, 0.24 mg/kg, 0.25 mg/kg, 0.26 mg/kg, 0.27 mg/kg, 0.28 mg/kg, 0.29 mg/kg, 0.3 mg/kg, 0.35 mg/kg, 0.4 mg/kg, 0.45 mg/kg, 0.5 mg/kg, 0.55 mg/kg, 0.6 mg/kg, 0.65 mg/kg, 0.7 mg/kg, 0.75 mg/kg, 0.8 mg/kg, 0.85 mg/kg, 0.9 mg/kg, 0.95 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, or 2 mg/kg.

In some embodiments, for each administration, the amount of rhGNS is from about 1 mg to about 100 mg per human patient. In some embodiments, for each administration, the amount of rhGNS is from about 1 mg to about 95 mg, from about 1 mg to about 90 mg, from about 1 mg to about 85 mg, from about 1 mg to about 80 mg, from about 1 mg to about 75 mg, from about 1 mg to about 70 mg, from about 1 mg to about 65 mg, from about 1 mg to about 60 mg, from about 1 mg to about 55 mg, from about 1 mg to about 50 mg, from about 1 mg to about 45 mg, from about 1 mg to about 40 mg, from about 1 mg to about 35 mg, from about 1 mg to about 30 mg, from about 1 mg to about 25 mg, from about 1 mg to about 20 mg, from about 1 mg to about 15 mg, from about 1 mg to about 10 mg, from about 5 mg to about 95 mg, from about 5 mg to about 90 mg, from about 5 mg to about 85 mg, from about 5 mg to about 80 mg, from about 5 mg to about 75 mg, from about 5 mg to about 70 mg, from about 5 mg to about 65 mg, from about 5 mg to about 60 mg, from about 5 mg to about 55 mg, from about 5 mg to about 50 mg, from about 5 mg to about 45 mg, from about 5 mg to about 40 mg, from about 5 mg to about 35 mg, from about 5 mg to about 30 mg, from about 5 mg to about 25 mg, from about 5 mg to about 20 mg, from about 5 mg to about 15 mg, from about 5 mg to about 10 mg, from about 10 mg to about 95 mg, from about 10 mg to about 90 mg, from about 10 mg to about 85 mg, from about 10 mg to about 80 mg, from about 10 mg to about 75 mg, from about 10 mg to about 70 mg, from about 10 mg to about 65 mg, from about 10 mg to about 60 mg, from about 10 mg to about 55 mg, from about 10 mg to about 50 mg, from about 10 mg to about 45 mg, from about 10 mg to about 40 mg, from about 10 mg to about 35 mg, from about 10 mg to about 30 mg, from about 10 mg to about 25 mg, from about 10 mg to about 20 mg, from about 10 mg to about 15 mg, from about 15 mg to about 95 mg, from about 15 mg to about 90 mg, from about 15 mg to about 85 mg, from about 15 mg to about 80 mg, from about 15 mg to about 75 mg, from about 15 mg to about 70 mg, from about 15 mg to about 65 mg, from about 15 mg to about 60 mg, from about 15 mg to about 55 mg, from about 15 mg to about 50 mg, from about 15 mg to about 45 mg, from about 15 mg to about 40 mg, from about 15 mg to about 35 mg, from about 15 mg to about 30 mg, from about 15 mg to about 25 mg, or from about 15 mg to about 20 mg.

In some embodiments, for each administration, the amount of rhGNS is from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 1 mg to about 10 mg, from about 5 mg to about 15 mg, from about 10 mg to about 20 mg, from about 15 mg to about 25 mg, from about 20 mg to about 30 mg, from about 25 mg to about 35 mg, from about 30 mg to about 40 mg, from about 35 mg to about 45 mg, from about 40 mg to about 50 mg, from about 50 mg to about 60 mg, from about 60 mg to about 70 mg, from about 70 mg to about 80, or from about 80 mg to about 90 mg.

In any of the methods described herein, it should be understood, even if not always explicitly stated, that an effective amount of an rhGNS of the present disclosure is administered to the subject. The amount can be empirically determined by the treating physician and will vary with the age, gender, weight and health of the subject. With these variables in mind, one of skill will administer a therapeutically effective amount to the subject to be treated. It is contemplated that a therapeutically effective amount of the rhGNS described herein may contain from about 0.01 milligram of rhGNS per kilogram of a subject's body weight to 1 gram of rhGNS per kilogram of a subject's body weight of rhGNS. In some aspects, a therapeutically effective amount of the rhGNS is from 50 mg to 2000 mg, or from 100 mg to 1000 mg, without limitation.

This GNS enzyme catalyzes the following chemical reaction: hydrolysis of the 6-sulfate groups of the N-acetyl-D-glucosamine 6-sulfate units of heparan sulfate and keratan sulfate. Therefore, in one embodiment, the enzymatic activity of human GNS protein refers to the ability to catalyze the hydrolysis of the 6-sulfate groups of the N-acetyl-D-glucosamine 6-sulfate units of heparan sulfate or keratan sulfate. Methods of measuring such an activity are well known in the art. In one embodiment, the rhGNS has at least 50% (or at least 60%, 70%, 80%, 85%, 90%, or 95%) activity of the wild-type human GNS in a suitable in vivo environment.

In some aspects, the composition is provided in an artificial cerebrospinal fluid. Methods of preparing artificial cerebrospinal fluids (ACSF) are known in the art and ACSF are also commercially available. The artificial cerebrospinal fluid may have a pH that is lower than about 8, lower than about 7.5, from about 5 to 8, from about 5.5 to about 7.5, from about 6 to about 7.5, from about 6 to about 7.

In some embodiments, the artificial cerebrospinal fluid comprises about 130-170 mEq/1 sodium, about 2.5-5 mEq/1 potassium, about 1-3 mEq/1 calcium, about 0.5-3 mEq/1 magnesium, about 120-180 mEq/1 chloride, and about 0.5-2 mEq/1 phosphate. In some embodiments, the artificial cerebrospinal fluid comprises about 140-160 mEq/1 sodium, about 3.5-4.5 mEq/1 potassium, about 2.5-3 mEq/1 calcium, about 2-3 mEq/1 magnesium, about 120-140 mEq/1 chloride, and about 1-2 mEq/1 phosphate. In some embodiments, the artificial cerebrospinal fluid comprises about 140-160 mEq/1 sodium, about 3.5-4.5 mEq/1 potassium, about 2.5-3 mEq/1 calcium, about 2-3 mEq/1 magnesium, about 120-140 mEq/1 chloride, about 1-2 mEq/1 phosphate, about 18-25 mEq/1 bicarbonate, and about 2-3 mEq/1 sulfate. The osmolarity of the artificial cerebrospinal fluid can be about 250-350 mOsm/1, or about 260-300 mOsm/1. In one aspect, the ACSF contains sodium 149 mEq/1, potassium 4 mEq/1, calcium 2.7 mEq/1, magnesium 2.4 mEq/1, bicarbonate 22.6 mEq/1, chloride 132 mEq/1, sulfate 2.4 mEq/1, phosphate 1.5 mEq/1, pH 6-7.5, 288 mOsm/1 but no protein.

In one embodiment, the rhGNS exhibits the maximum enzymatic activity at an acidic condition (e.g., pH 5.3 to 5.9, pH 5.4 to 5.8, pH 5.5 to 5.7, pH 5.55 to 5.65, or at about pH 5.6). In one embodiment, the rhGNS is at least twice as active at pH 5.6 as at pH 7.0. In another embodiment, the rhGNS is at least three times, four times, five times, six times, seven times, eight times, nine time or 10 times as active at pH 5.6 as at pH 7.0.

Method of obtaining rhGNS of high purity and activity are demonstrated in the experimental examples can such rhGNS may be useful for practice of certain embodiments of the invention. In one embodiment, a rhGNS composition suitable for the treatment includes from about 0.5 mg to about 30 mg of the recombinant protein per ml of the artificial cerebrospinal fluid, or from about 1 mg to about 25 mg, or from about 2 mg to about 20 mg, or from about 5 mg to about 15 mg per ml of the artificial cerebrospinal fluid.

In one embodiment, the rhGNS of the present disclosure is able to enter a human fibroblast cell when the recombinant polypeptide is incubated with the human fibroblast cell. In some aspects, such incubation does not involve the use of a cell penetrating peptide, a nanoparticle such as a liposome, and/or the assistant of an agent that induces cell endocytosis (or pinocytosis or phagocytosis).

In some aspects, the rhGNS of the present disclosure is suitably glycosylated. It is readily appreciated that recombinant protein expressed and prepared in an in vitro environment undergoes different glycosylation process and/or ends up with different glycosylation than its wild-type counterpart. In one aspect, the rhGNS of the present disclosure adds from 25 kDa to 45 kDa molecular weight to the recombinant polypeptide. In one aspect, the rhGNS of the present disclosure adds at least 20, 25, 30, 35, or 40 kDa molecular weight to the recombinant polypeptide. In one aspect, the rhGNS of the present disclosure adds not more than 30, 35, 40, 45, 50, 55, or 60 kDa molecular weight to the recombinant polypeptide. In one aspect, the rhGNS of the present disclosure adds at from 20 to 60, 25 to 55, 25 to 50, 25 to 45, or 25 to 40, or 25 to 35 kDa molecular weight to the recombinant polypeptide.

In some embodiments, the rhGNS is any one of the rhGNS as described above. In some embodiments, the rhGNS is conjugated with a moiety capable of extending the circulating half-life of the rhGNS. In some embodiments, the moiety is selected from the group consisting of polyethylene glycol, an acyl group, a liposome, a carrier protein, an artificial phospholipid membrane, and a nanoparticle.

A lysosomal targeting moiety can be added or conjugated to the rhGNS, in some embodiment, to facilitate delivery. Lysosomal targeting moieties are known in the art. In one embodiment, the targeting moiety is a means (e.g. a molecule) for binding the extracellular domain of the human cation-independent M6P receptor in an M6P-independent manner when the receptor is present in the plasma membrane of a target cell. In another embodiment, the targeting moiety is an unglycosylated lysosomal targeting domain that binds the extracellular domain of the human cation-independent M6P receptor. In either embodiment, the targeting moiety can include, for example, IGF-II; retinoic acid or a derivative thereof; a protein having an amino acid sequence at least 70% identical to a domain of urokinase-type plasminogen activator receptor; an antibody variable domain that recognizes the receptor; or variants thereof.

In another embodiment, the targeting moiety is a lysosomal targeting domain that binds the extracellular domain of the human cation-independent M6P receptor but does not bind a mutein of the receptor in which amino acid 1572 is changed from isoleucine to threonine, or binds the mutein with at least ten-fold less affinity (i.e. with at least a ten-fold greater dissociation constant). In another embodiment, the targeting moiety is a lysosomal targeting domain capable of binding a receptor domain consisting essentially of repeats 10-15 of the human cation-independent M6P receptor: the lysosomal targeting domain can bind a protein that includes repeats 10-15 even if the protein includes no other moieties that bind the lysosomal targeting domain. Preferably, the lysosomal targeting domain can bind a receptor domain consisting essentially of repeats 10-13 of the human cation-independent M6P receptor.

In some embodiments, the lysosomal targeting domain can bind a receptor domain consisting essentially of repeats 11-12, repeat 11, or amino acids 1508-1566 of the human cation-independent M6P receptor. In each of these embodiments, the lysosomal targeting domain preferably binds the receptor or receptor domain with a submicromolar dissociation constant at or about pH 7.4. In one preferred embodiment, the lysosomal targeting domain binds with an dissociation constant of about 10-7 M. In another preferred embodiment, the dissociation constant is less than about 10-7 M.

In another embodiment, the targeting moiety is a binding moiety sufficiently duplicative of human IGF-II such that the binding moiety binds the human cation-independent M6P receptor. The binding moiety can be sufficiently duplicative of IGF-II by including an amino acid sequence sufficiently homologous to at least a portion of IGF-II, or by including a molecular structure sufficiently representative of at least a portion of IGF-II, such that the binding moiety binds the cation-independent M6P receptor. The binding moiety can be an organic molecule having a three-dimensional shape representative of at least a portion of IGF-II, such as amino acids 48-55 of human IGF-II, or at least three amino acids selected from the group consisting of amino acids 8, 48, 49, 50, 54, and 55 of human IGF-II. A preferred organic molecule has a hydrophobic moiety at a position representative of amino acid 48 of human IGF-II and a positive charge at or about pH 7.4 at a position representative of amino acid 49 of human IGF-II. In one embodiment, the binding moiety is a polypeptide including a polypeptide having antiparallel alpha-helices separated by not more than five amino acids. In another embodiment, the binding moiety includes a polypeptide with the amino acid sequence of IGF-I or of a mutein of IGF-I in which amino acids 55-56 are changed and/or amino acids 1-4 are deleted or changed. In a further embodiment, the binding moiety includes a polypeptide with an amino acid sequence at least 60% identical to human IGF-II; amino acids at positions corresponding to positions 54 and 55 of human IGF-II are preferably uncharged or negatively charged at or about pH 7.4.

In one embodiment, the targeting moiety is a polypeptide comprising the amino acid sequence phenylalanine-arginine-serine. In another embodiment, the targeting moiety is a polypeptide including an amino acid sequence at least 75% homologous to amino acids 48-55 of human IGF-II. In another embodiment, the targeting moiety includes, on a single polypeptide or on separate polypeptides, amino acids 8-28 and 41-61 of human IGF-II. In another embodiment, the targeting moiety includes amino acids 41-61 of human IGF-II and a mutein of amino acids 8-28 of human IGF-II differing from the human sequence at amino acids 9, 19, 26, and/or 27.

### III. Methods of Preparing the Recombinant Human GNS Protein

Polypeptides of this disclosure can be prepared by expressing polynucleotides encoding the polypeptide sequences of this disclosure in an appropriate host cell. This can be accomplished by methods of recombinant DNA technology known to those skilled in the art. The proteins and polypeptides of this disclosure also can be obtained by chemical synthesis using a commercially available automated peptide synthesizer such as those manufactured by Perkin Elmer/Applied Biosystems, Inc., Model 430A or 431A, Foster City, Calif., USA. The synthesized protein or polypeptide can be precipitated and further purified, for example by high performance liquid chromatography (HPLC). Accordingly, this disclosure also provides a process for chemically synthesizing the proteins of this disclosure by providing the sequence of the protein and reagents, such as amino acids and enzymes and linking together the amino acids in the proper orientation and linear sequence.

It is known to those skilled in the art that modifications can be made to any peptide to provide it with altered properties. Polypeptides of the disclosure can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that α-helical secondary structure or random secondary structure is preferred.

In a further embodiment, subunits of polypeptides that confer useful chemical and structural properties will be chosen. For example, peptides comprising D-amino acids may be resistant to L-amino acid-specific proteases in vivo. Modified compounds with D-amino acids may be synthesized with the amino acids aligned in reverse order to produce the peptides of the disclosure as retro-inverso peptides. In addition, the present disclosure envisions preparing peptides that have better defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂NH—R₂, where R₁, and R₂ are amino acid residues or sequences. A reduced peptide bond may be introduced as a dipeptide subunit. Such a molecule would be resistant to peptide bond hydrolysis, e.g., protease activity. Such molecules would provide ligands with unique function and activity, such as extended half-lives in vivo due to resistance to metabolic breakdown, or protease activity. Furthermore, it is well known that in certain systems constrained peptides show enhanced functional activity (Hruby (1982) Life Sciences 31:189-199 and Hruby et al. (1990) Biochem J. 268:249-262); the present disclosure provides a method to produce a constrained peptide that incorporates random sequences at all other positions.

The following non-classical amino acids may be incorporated in the peptides of the disclosure in order to introduce particular conformational motifs: 1,2,3,4-tetrahydroisoquinoline-3-carboxylate (Kazrnierski et al. (1991) J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby (1991) Tetrahedron Lett. 32(41):5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1989) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (1993) Int. J. Pep. Protein Res. 42(1):68-77) and (Dharanipragada et al. (1992) Acta. Crystallogr. C. 48:1239-1241).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Lett. 29:5057-5060); α-helix inducing analogs (Kemp et al. (1988) Tetrahedron Lett. 29:4935-4938); α-turn inducing analogs (Kemp et al. (1989) J. Org. Chem. 54:109:115); analogs provided by the following references: Nagai and Sato (1985) Tetrahedron Lett. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Clones et al. (1988) Tetrahedron Lett. 29:3853-3856); tetrazole (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 56:436. Conformationally restricted mimetics of beta turns and beta bulges, and peptides containing them, are described in U.S. Pat. No. 5,440,013, issued Aug. 8, 1995 to Kahn.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobicity, size, or charge. Methods used to determine the appropriate amino acid to be substituted and for which amino acid are known to one of skill in the art. Non-limiting examples include empirical substitution models as described by Dahoff et al. (1978) In Atlas of Protein Sequence and Structure Vol. 5 suppl. 2 (ed. M. O. Dayhoff), pp. 345-352. National Biomedical Research Foundation, Washington D.C.; PAM matrices including Dayhoff matrices (Dahoff et al. (1978), supra, or JTT matrices as described by Jones et al. (1992) Comput. Appl. Biosci. 8:275-282 and Gonnet et al. (1992) Science 256:1443-1145; the empirical model described by Adach and Hasegawa (1996) J. Mol. Evol. 42:459-468; the block substitution matrices (BLOSUM) as described by Henikoff and Henikoff (1992) Proc. Natl. Acad. Sci. USA 89:10915-10919; Poisson models as described by Nei (1987) Molecular Evolutionary Genetics. Columbia University Press, New York.; and the Maximum Likelihood (ML) Method as described by Müller et al. (2002) Mol. Biol. Evol. 19:8-13.

### IV. Polynucleotides, Host Cells and Compositions

This disclosure also provides polynucleotides that encode any polypeptide of the present disclosure, and their complements. Complementarity can be determined using traditional hybridization under conditions of moderate or high stringency. As used herein, the term polynucleotide intends DNA and RNA as well as modified nucleotides. For example, this disclosure also provides the anti-sense polynucleotide stand, e.g. antisense RNA to these sequences or their complements.

Further provided, in one embodiment, are polynucleotide sequences useful for expressing the rhGNS protein. In one embodiment, the polynucleotide sequences are different from the wild-type human cDNA sequence, or any wild-type GNS sequence. In one embodiment, the coding sequence of GNS is optimized to achieve high expression efficiency. In one aspect, the coding sequence includes SEQ ID NO: 3.

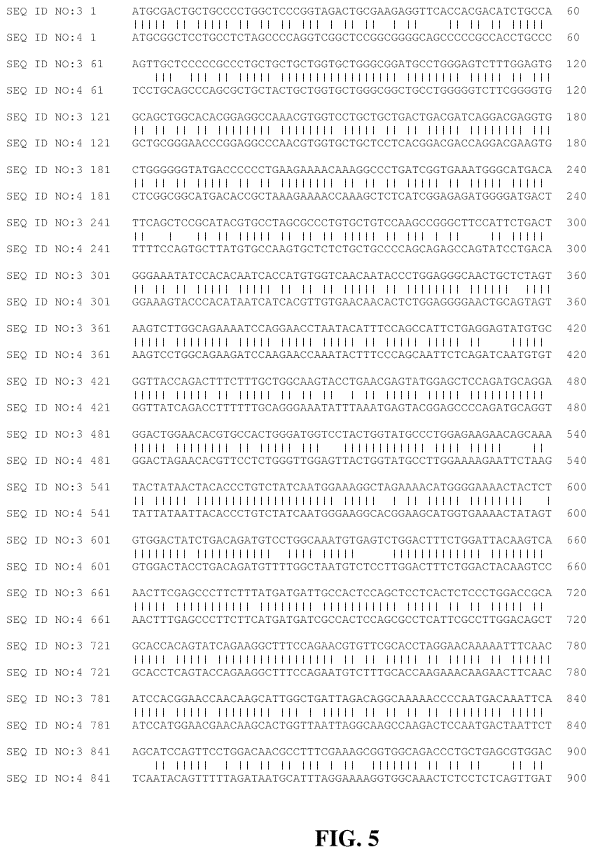

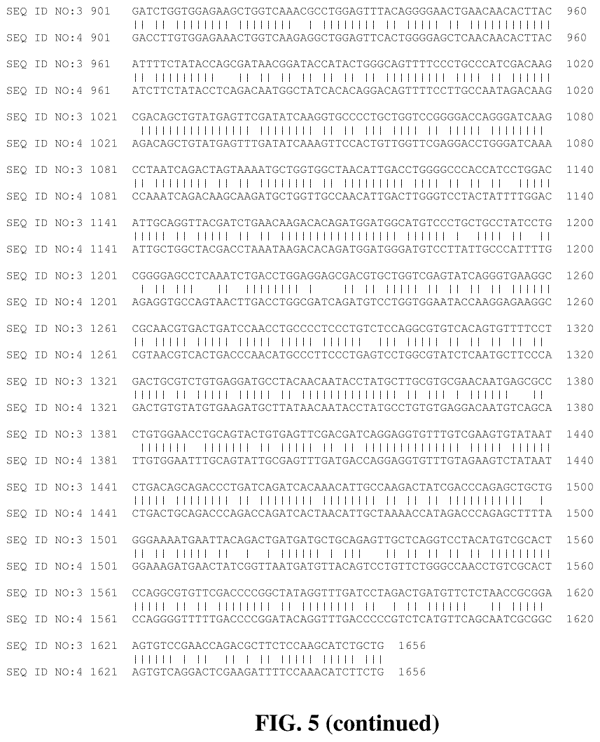

In some aspects, the polynucleotide includes a nucleic acid sequence (a) having at least 85% (or at least 75%, 80%, 90%, 95%, 97%, 98%, or 99%) sequence identify to SEQ ID NO: 3, (b) encoding the amino acid sequence of SEQ ID NO: 1 (or a biological equivalent of SEQ ID NO: 1 as disclosed herein), and (c) having no more than 95% (or no more than 90%, or 85%) sequence identity to SEQ ID NO: 4. As shown in FIG. 5, SEQ ID NO: 3 and 4 have a sequence identity of about 77.4% (1281 matches over 1656 nucleotides).

Also provided are polynucleotides encoding substantially homologous and biologically equivalent polypeptides to the inventive polypeptides and polypeptide complexes. Substantially homologous and biologically equivalent intends those having varying degrees of homology, such as at least 65%, or alternatively, at least 70%, or alternatively, at least 75%, or alternatively at least 80%, or alternatively, at least 85%, or alternatively at least 90%, or alternatively, at least 95%, or alternatively at least 97% homologous as defined above and which encode polypeptides having the biological activity of human GNS. It should be understood although not always explicitly stated that embodiments to substantially homologous polypeptides and polynucleotides are intended for each aspect of this disclosure, e.g., polypeptides, polynucleotides and antibodies.

The polynucleotides of this disclosure can be replicated using conventional recombinant techniques. Alternatively, the polynucleotides can be replicated using PCR technology. PCR is the subject matter of U.S. Pat. Nos. 4,683,195; 4,800,159; 4,754,065; and 4,683,202 and described in PCR: The Polymerase Chain Reaction (Mullis et al. eds, Birkhauser Press, Boston (1994)) and references cited therein. Yet further, one of skill in the art can use the sequences provided herein and a commercial DNA synthesizer to replicate the DNA. Accordingly, this disclosure also provides a process for obtaining the polynucleotides of this disclosure by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can operatively link the polynucleotides to regulatory sequences for their expression in a host cell. The polynucleotides and regulatory sequences are inserted into the host cell (prokaryotic or eukaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable prokaryotic or eukaryotic host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook and Russell (2001) supra. For instance, mRNA can be isolated using various lytic enzymes or chemical solutions according to the procedures set forth in Sambrook and Russell (2001) supra or extracted by nucleic-acid-binding resins following the accompanying instructions provided by manufactures. In one embodiment, provided is a construct comprising the polynucleotide, a protein prepared by expressing the polynucleotide, a cell enclosing the polynucleotide, or a cell stably transfected with the polynucleotide, which is optionally integrated into the cell chromosomes.

Also provided are host cells comprising one or more of the polypeptides or polynucleotides of this disclosure. In one aspect, the polypeptides are expressed and present on the cell surface (extracellularly). Suitable cells containing the disclosed polypeptides include prokaryotic and eukaryotic cells, which include, but are not limited to bacterial cells, yeast cells, insect cells, animal cells, mammalian cells, murine cells, rat cells, sheep cells, simian cells and human cells. Examples of bacterial cells include Escherichia coli, Salmonella enterica and Streptococcus gordonii. The cells can be purchased from a commercial vendor such as the American Type Culture Collection (ATCC, Rockville Md., USA) or cultured from an isolate using methods known in the art. Examples of suitable eukaryotic cells include, but are not limited to 293T HEK cells, as well as the hamster cell line CHO, BHK-21; the murine cell lines designated NIH3T3, NS0, C127, the simian cell lines COS, Vero; and the human cell lines HeLa, PER.C6 (commercially available from Crucell) U-937 and Hep G2. A non-limiting example of insect cells include Spodoptera frugiperda. Examples of yeast useful for expression include, but are not limited to Saccharomyces, Schizosaccharomyces, Hansenula, Candida, Torulopsis, Yarrowia, or Pichia. See e.g., U.S. Pat. Nos. 4,812,405; 4,818,700; 4,929,555; 5,736,383; 5,955,349; 5,888,768 and 6,258,559.

The present disclosure further provides compositions comprising an rhGNS of the present disclosure and a pharmaceutically acceptable carrier.

“Pharmaceutically acceptable carriers” refers to any diluents, excipients, or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

The pharmaceutical compositions of the disclosure can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Formulations may optionally contain stabilizers, pH modifiers, surfactants, bioavailability modifiers and combinations of these.

Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The compositions of this disclosure are formulated for pharmaceutical administration to a mammal, preferably a human being. Such pharmaceutical compositions of the disclosure may be administered in a variety of ways, preferably intrathecally. Other routes, such as intravenous and intranasal are contemplated as well.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed is an artificial cerebrospinal fluid. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

In addition to dosage forms described above, pharmaceutically acceptable excipients and carriers and dosage forms are generally known to those skilled in the art and are included in the disclosure. It should be understood that a specific dosage and treatment regimen for any particular patient will depend upon a variety of factors, including the activity of the specific rhGNS employed, the age, body weight, general health, sex and diet, renal and hepatic function of the patient, and the time of administration, rate of excretion, drug combination, judgment of the treating physician or veterinarian and severity of the particular disease being treated.

Also provided by this disclosure are pharmaceutical compositions containing one or more of the rhGNS of the present disclosure and a pharmaceutically acceptable carrier. The compositions are administered to a subject in need thereof in an amount that will provide the desired benefit. The compositions can be co-administered with any suitable agent or therapy that complements or enhances the activity of the rhGNS. An example of such is a second agent capable of extending the plasma half-life of the rhGNS. Examples of suitable second agents include but are not limited to an anti-rhGNS antibody recognizing the exosite of the rhGNS.

Combination therapies are also provided. In addition to the injection, the patient can further receive a therapy such as bone marrow replacement, or administration of genistein or a chaperone. Genistein is a compound with the chemical name of 5,7-Dihydroxy-3-(4-hydroxyphenyl)chromen-4-one. Suitable chaperones can be screened by high throughput screening and computational screening for a particular patient.

### EXAMPLES

The disclosure is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1. Preparation and In Vitro Testing of rhGNS

In this example, a stably-transfected Chinese hamster ovarian (CHO) cell line was used to produce pre-clinical levels of recombinant human GNS (rhGNS) protein. rhGNS has been purified and enzymatically characterized. Suitable storage conditions have been identified for both longevity and safe administration. Using MPS IIID fibroblasts, this example has evaluated its cellular uptake, mediated via the M6P receptor, and further demonstrated localization within the lysosome and the ability to reduce glycosaminoglycan (GAG) storage.

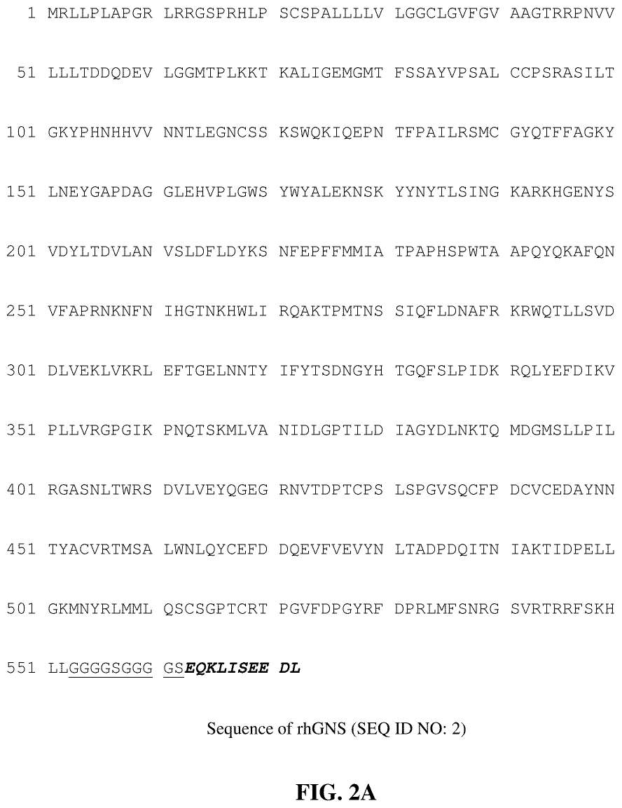

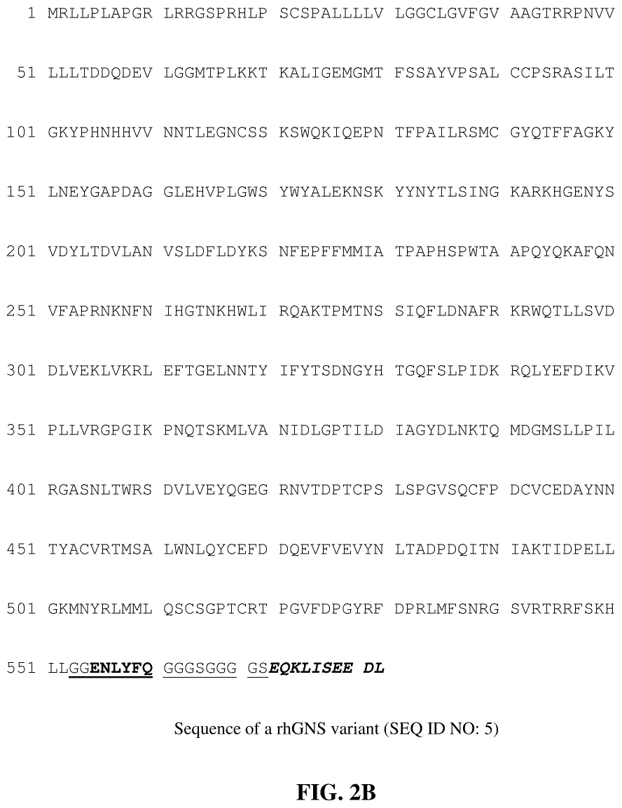

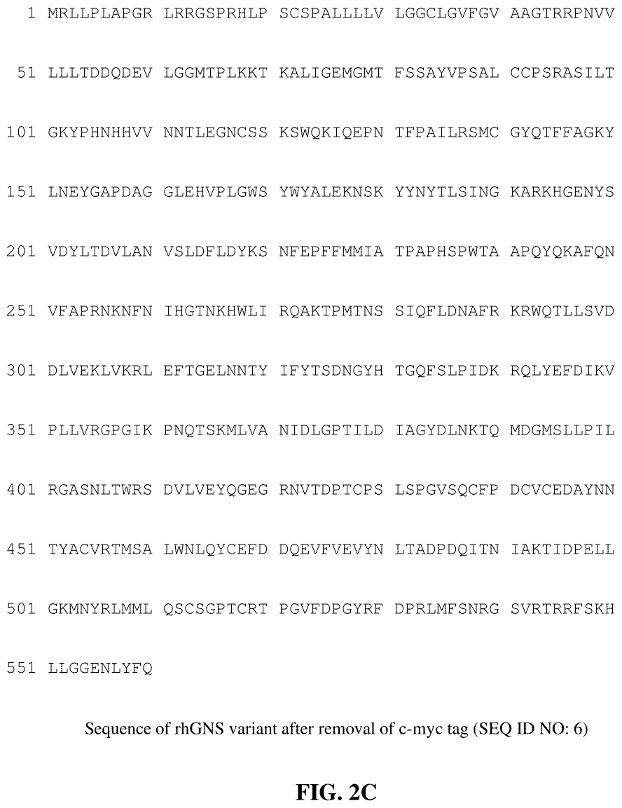

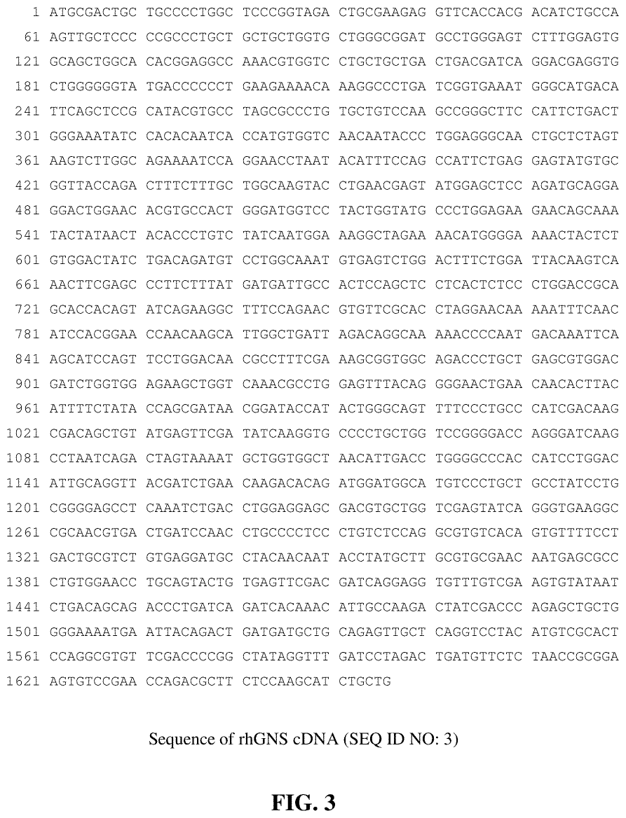

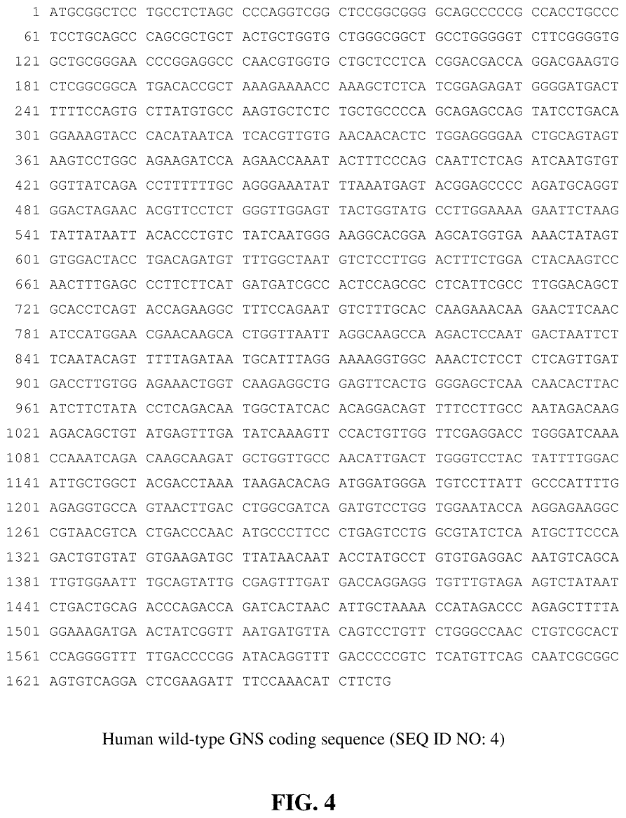

The coding sequence (cDNA) of rhGNS (SEQ ID NO: 3, FIG. 3; for comparison, see the wild-type sequence of SEQ ID NO: 4, FIG. 4, and their sequence alignment in FIG. 5) was inserted into a mammalian expression plasmid using restriction enzyme digestion and ligation. The rhGNS can contain a c-myc moiety (as illustrated in FIG. 2A) or contains a C-terminal TEV protease cleavage site between the protein and the c-myc moiety (see FIGS. 2B and 2C for pre- and post-cleavage sequences) for ease of purification, and expression is driven by a CMV promoter. Chinese hamster ovary (CHO) cells were stably transfected, isolated, and screened for high expressing clones. Cells were grown in roller bottles and media harvested to obtain the secreted rhGNS. Following concentration, media was loaded into a c-myc affinity column, washed, and then eluted using soluble c-myc peptide in artificial cerebrospinal fluid (Elliotts B Solution, USP). Eluted rhGNS was then concentrated to a final concentration of 1 mg/ml. This example achieved a yield of >100 μg rhGNS per 1500 mL media, reaching a specific activity >200,000 units/mg (Table 1).

TABLE 1
Purification of secreted rhGNS from CHO cell line
using c-myc affinity column.

| | Volume | Protein | Total activity | Specific Activity |
|---|---|---|---|---|
| Step | (ml) | (μg) | (units)* | (units/mg protein) |
| PF CHO Media | 1,450 | 330,165 | 108,998 | 330 |
| Concentrated | 110 | 304,920 | 307,826 | 1,010 |
| Media | | | | |
| Flow through | 110 | 302,878 | 273,667 | 904 |
| myc Elution | 3 | 186 | 26,408 | 142,263 |
| Elution | 0.14 | 119 | 28,593 | 241,058 |
| Concentration | | | | |

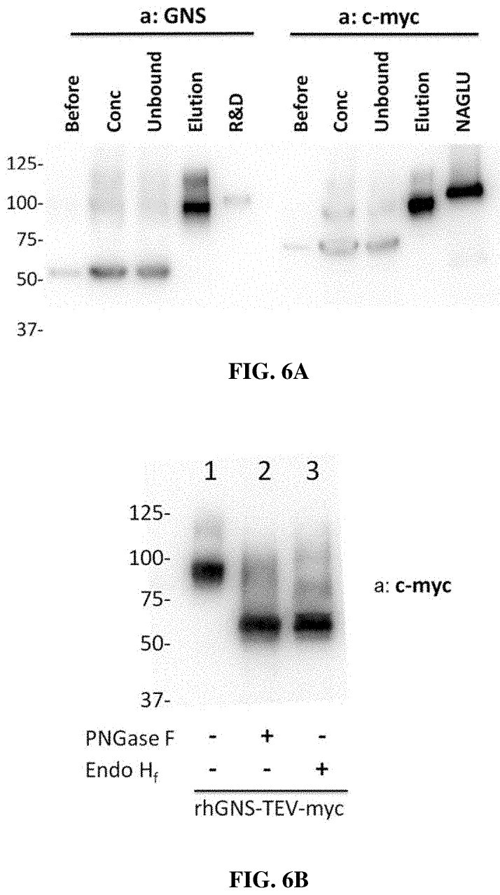

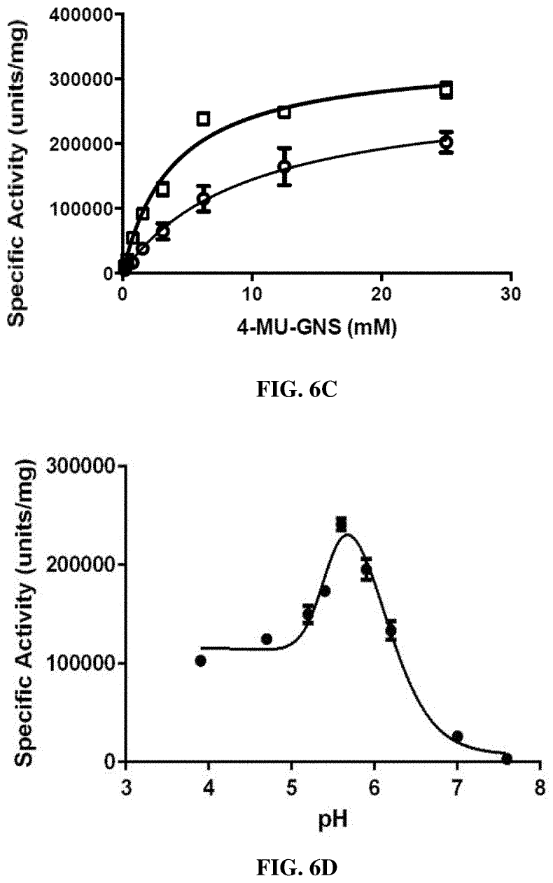

Western Blot analysis of the purification steps (FIG. 6A) and glycosidase digestion revealed that purified rhGNS is highly glycosylated (FIG. 6B), which is vital for both intracellular uptake and lysosomal targeting. Using the fluorogenic substrate 4-Methylumbelliferyl 6-Sulfo-2-acetamido-2-deoxy-α-D-glucopyranoside (4-MU-GNS), purified rhGNS was shown to be both enzymatically active and stable following storage in artificial cerebral spinal fluid (FIG. 6C). Further biochemical characterization of the enzyme showed optimal reaction conditions within the lysosomal pH range (4-5.6), with 10-fold lower activity at neutral pH (FIG. 6D).

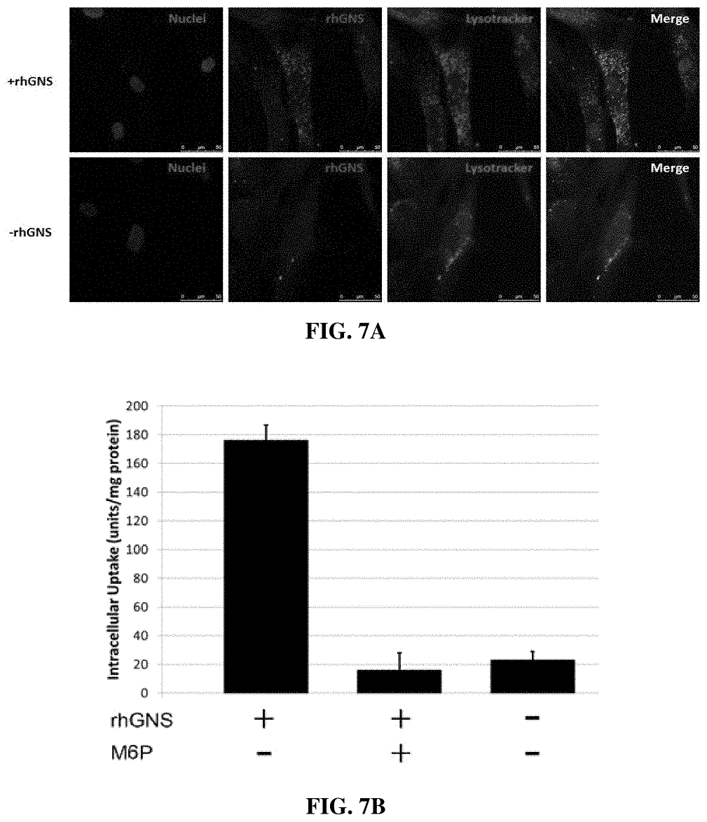

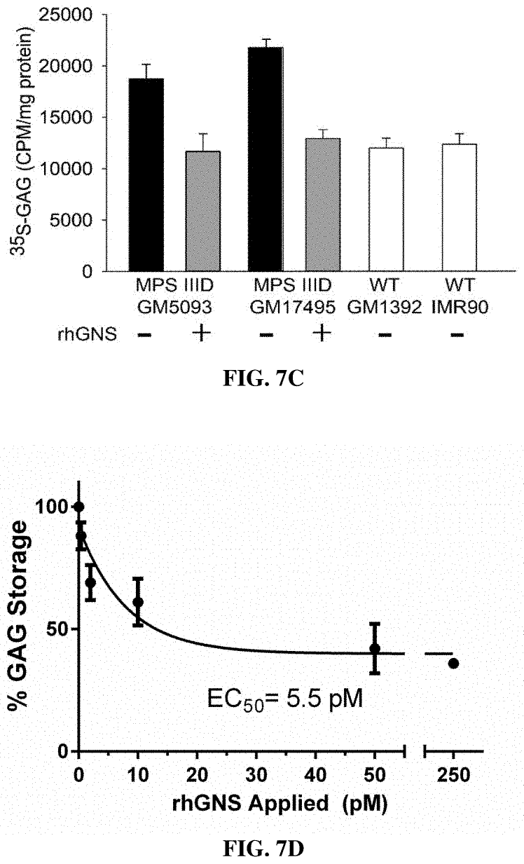

Enzymatically-active rhGNS was taken up by MPSIIID fibroblasts and co-localized with lysosomal markers (FIG. 7A). Both uptake and lysosomal targeting were shown to be decrease significantly in the presence of free M6P (FIG. 7B), suggesting that our rhGNS is rich in M6P glycosylation and uptake is M6P receptor dependent. We demonstrated a minimum of 33% and a maximum of 65% reduction in heparan sulfate in three independent experiments in two human MPS IIID cell lines treated with rhGNS, reaching wild-type levels of heparan sulfate (FIG. 7C). Further, radio-labeled GAG accumulation measured at different concentrations of purified rhGNS (0-250 pM) demonstrated an exponential decrease in storage, with storage reduced by half (EC₅₀) at 5.5 pM. Radiolabeled GAGs were extracted and measured via scintillation counting, and radioactive counts per minute were normalized to protein concentration (FIG. 7D). This result indicates that rhGNS produced in this example was not only active against the artificial substrate, but was also able to catabolize the primary substrate that is responsible for MPS IIID neuropathology.

Rare childhood neurodegenerative disorders like MPS IIID are some of the most heartbreaking and devastating diseases imaginable. To date, there is no approved treatment or cure for MPS IIID. Using a CHO stable cell line, this example was able to produce and purify enzymatically active and highly-glycosylated rhGNS. The product was mannose-6-phosphorylated, entered into human MPS IIID cells, targets to the lysosomal compartment, and is stable in the ideal vehicle for intrathecal delivery. Furthermore, we have shown that it is able to decrease accumulation of GAG in MPS IIID patient fibroblasts to WT levels, thereby correcting the primary physiological effects of the disease.

### Example 2. Additional Testing of rhGNS

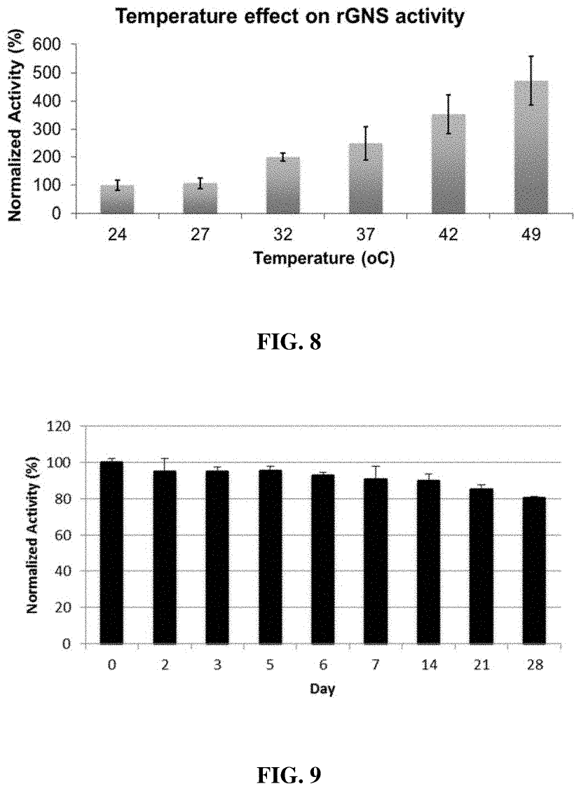

As shown in FIG. 8, this example assayed rhGNS over a range of temperature and found good enzymatic activity at body temperature.

The example also developed a storage buffer that will enable at least 1 month of storage. Purified rhGNS was tested in a variety of buffers, and was found to be stable for over one month at 4° C. in artificial cerebrospinal fluid (FIG. 9). Artificial cerebrospinal fluid is formulated to mimic the electrolyte composition of natural cerebrospinal fluid but contains no protein (sodium 149 mEq/1, potassium 4 mEq/1, calcium 2.7 mEq/1, magnesium 2.4 mEq/1, bicarbonate 22.6 mEq/1, chloride 132 mEq/1, sulfate 2.4 mEq/1, phosphate 1.5 mEq/1, pH 6-7.5, 288 mOsm/1). Thus not only did this example demonstrate stability of rhGNS for >1 month in a storage buffer, it was able to show stability in the ideal storage buffer for intrathecal administration to patients.

### Example 3. In Vivo Testing of rhGNS

This example will perform in vivo proof-of-concept studies in MPS IIID mice. In vivo effectiveness of the intrathecal delivery of rhGNS can be demonstrated such as the alleviation of neurologic, cognitive, and/or neurobehavioral pathologies caused by MPS IIID. This example will scale up our production of rhGNS so that the enzyme activity, lysosomal storage reduction, neuropathology and half-life estimation can be studied in the recently characterized MPS IIID knock out mouse. It is contemplated that this example will produce sufficient rhGNS, demonstrate increased enzyme activity, leading to lysosomal storage reduction and improved neuropathology and a half-life in the MPS IIID knock out mouse that would ultimately be suggestive of favorable kinetics in humans.

This example will also perform process development and product characterization. Eventual production of rhGNS for preclinical studies, clinical trials, and human patients will require a scalable process that can be readily adapted to a current good manufacturing practice (CGMP) facility. This example will develop a production and purification process that is scalable to preclinical and clinical needs, and perform product characterization (protein interaction, aggregation, glycosylation, etc.) and assessment of batch-to-batch variability.

### Example 4. Testing of rhGNS in MPS IIID Mice

This example tested the efficacy of rhGNS in MPS IIID knock out mice, with non-diseased mice as control. Recombinant human GNS (rhGNS) were produced as described above, using cerebrospinal fluid (CSF) as the vehicle which alone served as control. The treatment groups are shown in the table below.

| Group No. (Name) | Mice | Treatment | n |
|---|---|---|---|
| 1 (Carrier) | Non-diseased | CSF | 3 |
| 2 (MPS 3D) | MPS IIID | CSF | 3 |
| 3 (MPS 3D ERT) | MPS IIID | rhGNS (5.3 μg) | 2 |

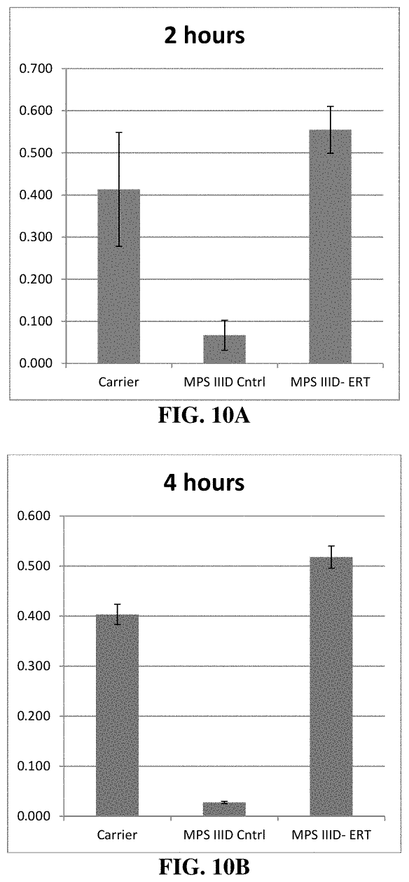

As shown in FIG. 10A, only two hours after dosing, the GNS enzyme activity (nmol/hr/mg) in group 3 was already higher than in the control group 1 (carrier). By contrast, group 2 which did not receive the treatment had hardly detectable GNS activity. Similar results were also observed at 4 hours (FIG. 10B).

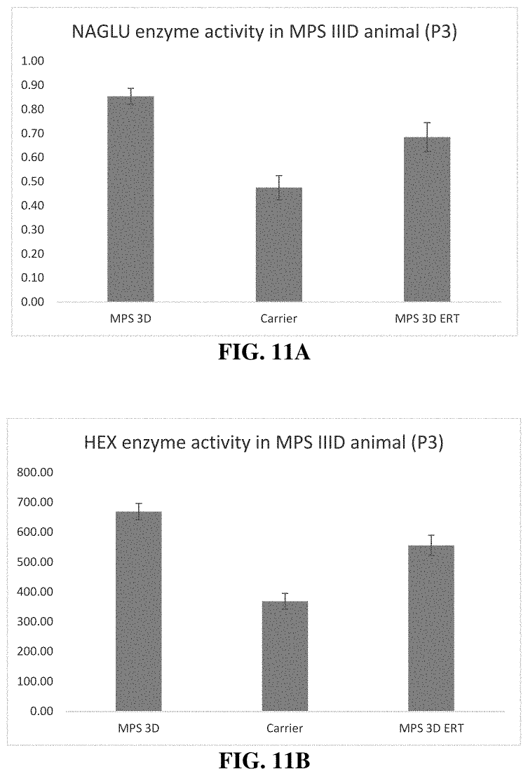

The activities of two lysosomal enzymes, alpha-N-acetylglucosaminidase (NAGLU) and β-hexoaminidase (HEX), were measured one day after dosing. The results are presented in FIGS. 11A and 11B, respectively. For both enzymes, the activity levels were higher in MPS IIID mice, but the activities were reduced by the treatment, demonstrating the effectiveness of the treatment.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method of treating mucopolysaccharidosis type IIID (MPS IIID) in a human patient in need thereof, comprising injecting to the spinal fluid of the patient an effective amount of a composition comprising a recombinant polypeptide comprising the amino acid sequence of SEQ ID NO: 1 or an amino acid sequence (a) having at least 95% sequence identity to SEQ ID NO: 1 and (b) having the enzymatic activity of human acetylglucosamine-6-sulfatase (GNS), wherein the composition is provided in an artificial cerebrospinal fluid.

2. The method of claim 1, wherein about 1 mg to about 100 mg of the recombinant polypeptide is administered to the patient once every 2 to 26 weeks.

3. The method of claim 1, wherein about 5 mg to about 30 mg of the recombinant polypeptide is administered to the patient once every 4 to 26 weeks.

4. The method of claim 1, wherein about 10 mg to about 20 mg of the recombinant polypeptide is administered to the patient once every 4 to 26 weeks.

5. The method of claim 1, wherein about 10 mg to about 20 mg of the recombinant polypeptide is administered to the patient once every 8 to 26 weeks.

6. The method of claim 5, wherein the recombinant polypeptide comprises the amino acid sequence of SEQ ID NO: 2, 5 or 6.

7. The method of claim 1, wherein the artificial cerebrospinal fluid has a pH of about 6 to 7.5.

8. The method of claim 5, wherein the artificial cerebrospinal fluid comprises:
about 130-170 mEq/1 sodium,
about 2.5-5 mEq/1 potassium,
about 1-3 mEq/1 calcium,
about 0.5-3 mEq/1 magnesium,
about 120-180 mEq/1 chloride, and
about 0.5-2 mEq/1 phosphate.

9. The method of claim 5, wherein the artificial cerebrospinal fluid comprises:
about 140-160 mEq/1 sodium,
about 3.5-4.5 mEq/1 potassium,
about 2.5-3 mEq/1 calcium,
about 2-3 mEq/1 magnesium,
about 120-140 mEq/1 chloride, and
about 1-2 mEq/1 phosphate.

10. The method of claim 5, wherein the artificial cerebrospinal fluid comprises:
about 140-160 mEq/1 sodium,
about 3.5-4.5 mEq/1 potassium,
about 2.5-3 mEq/1 calcium,
about 2-3 mEq/1 magnesium,
about 120-140 mEq/1 chloride,
about 1-2 mEq/1 phosphate,
about 18-25 mEq/1 bicarbonate, and
about 2-3 mEq/1 sulfate.

11. The method of claim 8, wherein the artificial cerebrospinal fluid has an osmolarity of about 250-350 mOsm/1.

12. The method of claim 1, wherein the recombinant polypeptide has maximum enzymatic activity at a pH within 5.4 to 5.8.

13. The method of claim 1, wherein composition comprises from about 0.5 mg to about 30 mg of the recombinant protein per ml of the artificial cerebrospinal fluid.

14. The method of claim 1, wherein the recombinant polypeptide enters a human fibroblast cell when the recombinant polypeptide is incubated with the human fibroblast cell.

15. The method of claim 1, wherein the recombinant polypeptide further comprises a lysosomal targeting moiety.

16. The method of claim 1, wherein the recombinant polypeptide is glycosylated, which glycosylation adds from 25 kDa to 45 kDa molecular weight to the recombinant polypeptide.

17. The method of claim 1, further comprising applying a second therapy to the patient.

18. The method of claim 17, wherein the second therapy comprises a bone marrow replacement, or administration of genistein or a chaperone.
